(19) United States
(12) Patent Application Publication
(10) Pub. No.: US 2023/0037360 A1
(43) Pub. Date: Feb. 9, 2023

(54) SYSTEMS AND METHODS FOR DETERMINING A SLEEP TIME

(71) Applicant: ResMed Sensor Technologies Limited, Dublin (IE)

(72) Inventors: Cesar Lopes, Dublin (IE); Niall O'Mahony, Dublin (IE); Michael Wren, Dublin (IE); Redmond Shouldice, Dublin (IE)

(21) Appl. No.: 17/789,120

(22) PCT Filed: Dec. 24, 2020
(86) PCT No.: PCT/IB2020/062427

§ 371 Date: Jun. 24, 2022

Related U.S. Application Data
(60) Provisional application No. 62/955,960, filed on Dec. 31, 2019.

(51) Int. Cl. A61B 5/00; G16H 20/70

(52) U.S. Cl. CPC A61B 5/4806; A61B 5/7267; A61B 5/486; G16H 20/70; A61B 2562/06

(57) ABSTRACT

A method includes receiving first physiological data associated with a user during a first sleep session. The method also includes receiving second physiological data associated with the user subsequent to the first sleep session and prior to a second sleep session. The method also includes determining a recommended bedtime for the user for the second sleep session based at least in part on the first physiological data, the second physiological data, or both. The method also includes causing an indication of the recommended bedtime for the second sleep session to be communicated to the user via a user device before the recommended bedtime.

### CROSS-REFERENCE TO RELATED APPLICATIONS

This application claims the benefit of, and priority to, U.S. Provisional Patent Application No. 62/955,960, filed Dec. 31, 2019, which is hereby incorporated by referenced herein in its entirety.

### TECHNICAL FIELD

The present disclosure relates generally to systems and methods for determining a recommended bedtime for a user, and more particularly, to systems and methods for determining a recommended bedtime for a sleep session and communicating the recommended bedtime to the user before the recommended bedtime.

### BACKGROUND

Many individuals suffer from insomnia (e.g., difficulty initiating sleep, frequent or prolonged awakenings after initially falling asleep, and an early awakening with an inability to return to sleep) or other sleep-related disorders (e.g., periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), etc.). Many of these sleep related disorders can be treated or managed if the individual goes to bed at an optimal time each night, wakes up at an optimal time, and/or sleeps for an optimal duration. Thus, it would be advantageous to determine a recommended bedtime for a user and communicate that recommended bedtime to the user to encourage the user to go to bed at the recommended time. The present disclosure is directed to solving these and other problems.

### SUMMARY

According to some implementations of the present disclosure, a method includes receiving first physiological data associated with a user during a first sleep session. The method also includes receiving second physiological data associated with the user subsequent to the first sleep session and prior to a second sleep session. The method also includes determining a recommended bedtime for the user for the second sleep session based at least in part on the first physiological data, the second physiological data, or both. The method also includes causing an indication of the recommended bedtime for the second sleep session to be communicated to the user via a user device before the recommended bedtime.

According to some implementations of the present disclosure, a system includes a first sensor, a second sensor, a memory, and a control system. The first sensor is configured to generate first physiological data associated with a user during a first sleep session. The second sensor configured to generate second physiological data associated with the user subsequent to the first sleep session and prior to a second sleep session. The memory stores machine-readable instructions. The control system includes one or more processors configured to execute the machine-readable instructions to determine a recommended bedtime for the user for the second sleep session based at least in part on the first physiological data, the second physiological data, or both. The control system is further configured to cause an indication of the recommended bedtime for the second sleep session to be communicated to the user via a user device before the recommended bedtime.

According to some implementations of the present disclosure, a system includes a sensor, a memory, and a control system. The sensor is configured to generate physiological data associated with a user during a plurality of sleep sessions. The memory stores machine-readable instructions. The control system includes one or more processors configured to execute the machine-readable instructions to accumulate historical physiological data for the user including previously recorded physiological data for the plurality of sleep sessions. The control system is further configured to receive information indicative of a sleep objective for a next sleep session. The control system is further configured to determine a recommended bedtime for the next sleep session based at least in part on the accumulated historical physiological data for the plurality of sleep sessions and the received sleep objective.

According to some implementations of the present disclosure, a method includes receiving first physiological data associated with a user during a plurality of sleep sessions, the plurality of sleep sessions including one or more pairs of successive sleep sessions. The method also includes receiving second physiological data associated with the user, the second physiological data being generated between each of the one or more pairs of successive sleep sessions, the second physiological data including historical second physiological data and current second physiological data. The method also includes determining a recommended bedtime for the user for a next sleep session using a machine learning algorithm based at least in part on the current second physiological data.

According to some implementations of the present disclosure, a system includes a first sensor, a second sensor, a memory, and a control system. The first sensor is configured to generate first physiological data associated with a user during a plurality of sleep sessions, the plurality of sleep sessions including one or more pairs of sleep sessions. The second sensor is configured to generate second physiological data associated with the user, the second physiological data being generated between each of the one or more pairs of sleep sessions. The memory stores machine-readable instructions. The control system includes one or more processors configured to execute the machine-readable instructions to accumulate the first physiological data and the second physiological data, the second physiological data including historical second physiological data and current second physiological data. The control system is further configured to train a machine learning algorithm with the first physiological data and the historical second physiological data such that the machine learning algorithm is configured to (i) receive as an input the current second physiological data and (ii) determine as an output a recommended bedtime for the user for a next sleep session.

According to some implementations of the present disclosure, a system includes a first sensor, a second sensor, a memory, and a control system. The first sensor is configured to generate first physiological data associated with a user during a plurality of sleep sessions, the plurality of sleep sessions including one or more pairs of sleep sessions. The second sensor is configured to generate second physiological data associated with the user, the second physiological data being generated between each of the one or more pairs of sleep sessions. The memory stores machine-readable instructions. The control system includes one or more processors configured to execute the machine-readable instructions to accumulate the first physiological data and the second physiological data, the second physiological data including historical second physiological data and current second physiological data. The control system is further configured to train a machine learning algorithm based on the first physiological data and the historical second physiological data such that the machine learning algorithm is configured to (i) receive as an input the current second physiological data, (ii) determine as a first output a recommended bedtime for the user for a next sleep session, and (iii) determine as a second output a reminder time prior to the recommended bedtime for causing an indication the recommended bedtime to be communicated to the user via a user device.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system for determining a recommended bedtime for a sleep session, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5 is a process flow diagram for a method of determining a recommended bedtime for a user, according to some implementations of the present disclosure;

FIG. 6A illustrates a first visual indicator of a recommended bedtime on a display device, according to some implementations of the present disclosure;

FIG. 6B illustrates a second visual indicator of a recommended bedtime on a display device, according to some implementations of the present disclosure;

FIG. 6C illustrates a user interface displayed on a display device for receiving a desired wake-up time from a user, according to some implementations of the present disclosure;

FIG. 7 is a process flow diagram for a method of determining a joint recommended bedtime for a user and a bedpartner of the user, according to some implementations of the present disclosure;

FIG. 8 is a process flow diagram for a method of determining a recommended bedtime for a next sleep session, according to some implementations of the present disclosure;

FIG. 9 is a process flow diagram for a method of determining a recommended bedtime for a next sleep session using a trained machine learning algorithm, according to some implementations of the present disclosure; and

FIG. 10 is a process flow diagram for a method of determining a recommended bedtime and determining a reminder time using a trained machine learning algorithm, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from insomnia, a condition which is generally characterized by a dissatisfaction with sleep quality or duration (e.g., difficulty initiating sleep, frequent or prolonged awakenings after initially falling asleep, and an early awakening with an inability to return to sleep). It is estimated that over 2.6 billion people worldwide experience some form of insomnia, and over 750 million people worldwide suffer from a diagnosed insomnia disorder. In the United States, insomnia causes an estimated gross economic burden of $107.5 billion per year, and accounts for 13.6% of all days out of role and 4.6% of injuries requiring medical attention. Recent research also shows that insomnia is the second most prevalent mental disorder, and that insomnia is a primary risk factor for depression.

Nocturnal insomnia symptoms generally include, for example, reduced sleep quality, reduced sleep duration, sleep-onset insomnia, sleep-maintenance insomnia, late insomnia, mixed insomnia, and/or paradoxical insomnia. Sleep-onset insomnia is characterized by difficulty initiating sleep at bedtime. Sleep-maintenance insomnia is characterized by frequent and/or prolonged awakenings during the night after initially falling asleep. Late insomnia is characterized by an early morning awakening (e.g., prior to a target or desired wakeup time) with the inability to go back to sleep. Comorbid insomnia refers to a type of insomnia where the insomnia symptoms are caused at least in part by a symptom or complication of another physical or mental condition (e.g., anxiety, depression, medical conditions, and/or medication usage). Mixed insomnia refers to a combination of attributes of other types of insomnia (e.g., a combination of sleep-onset, sleep-maintenance, and late insomnia symptoms). Paradoxical insomnia refers to a disconnect or disparity between the user's perceived sleep quality and the user's actual sleep quality.

Diurnal (e.g., daytime) insomnia symptoms include, for example, fatigue, reduced energy, impaired cognition (e.g., attention, concentration, and/or memory), difficulty functioning in academic or occupational settings, and/or mood disturbances. These symptoms can lead to psychological complications such as, for example, lower performance, decreased reaction time, increased risk of depression, and/or increased risk of anxiety disorders. Insomnia symptoms can also lead to physiological complications such as, for example, poor immune system function, high blood pressure, increased risk of heart disease, increased risk of diabetes, weight gain, and/or obesity.

Insomnia can also be categorized based on its duration. For example, insomnia symptoms are considered acute or transient if they occur for less than 3 months. Conversely, insomnia symptoms are considered chronic or persistent if they occur for 3 months or more, for example. Persistent/chronic insomnia symptoms often require a different treatment path than acute/transient insomnia symptoms.

Mechanisms of insomnia include predisposing factors, precipitating factors, and perpetuating factors. Predisposing factors include hyperarousal, which is characterized by increased physiological arousal during sleep and wakefulness. Measures of hyperarousal include, for example, increased levels of cortisol, increased activity of the autonomic nervous system (e.g., as indicated by increase resting heart rate and/or altered heart rate), increased brain activity (e.g., increased EEG frequencies during sleep and/or increased number of arousals during REM sleep), increased metabolic rate, increased body temperature and/or increased activity in the pituitary-adrenal axis. Precipitating factors include stressful life events (e.g., related to employment or education, relationships, etc.) Perpetuating factors include excessive worrying about sleep loss and the resulting consequences, which may maintain insomnia symptoms even after the precipitating factor has been removed.

Once diagnosed, insomnia can be managed or treated using a variety of techniques or providing recommendations to the patient. Generally, the patient can be encouraged or recommended to generally practice healthy sleep habits (e.g., plenty of exercise and daytime activity, have a routine, no bed during the day, eat dinner early, relax before bedtime, avoid caffeine in the afternoon, avoid alcohol, make bedroom comfortable, remove bedroom distractions, get out of bed if not sleepy, try to wake up at the same time each day regardless of bed time) or discouraged from certain habits (e.g., do not work in bed, do not go to bed too early, do not go to bed if not tired). An individual suffering from insomnia can be treated by improving the sleep hygiene of the individual. Sleep hygiene generally refers to the individual's practices (e.g., die, exercise, substance use, bedtime, activities before going to sleep, activities in bed before going to sleep, etc.) and/or environmental parameters (e.g., ambient light, ambient noise, ambient temperature, etc.). In at least some cases, the individual can improve their sleep hygiene by going to bed at a certain bedtime each night, sleeping for a certain duration, waking up at a certain time, modifying the environmental parameters, or any combination thereof.

Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood. Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness. Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping. While these other sleep-related disorders may have similar symptoms as insomnia, distinguishing these other sleep-related disorders from insomnia is useful for tailoring an effective treatment plan distinguishing characteristics that may call for different treatments. For example, fatigue is generally a feature of insomnia, whereas excessive daytime sleepiness is a characteristic feature of other disorders (e.g., PLMD) and reflects a physiological propensity to fall asleep unintentionally.

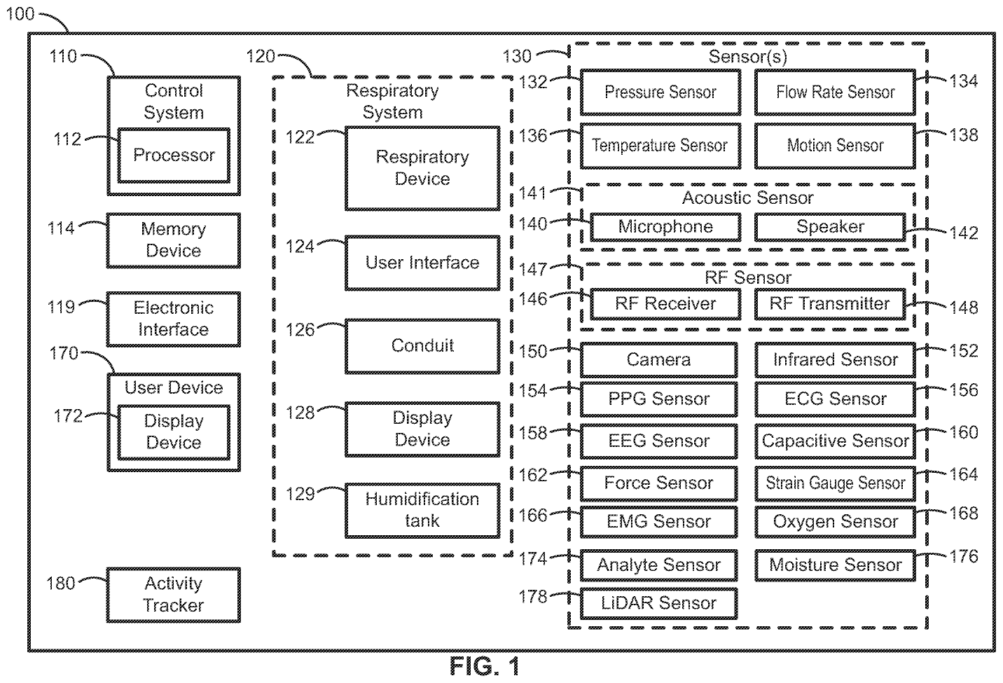

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory system 120, and an activity tracker 180.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory pressure therapy device 122 (referred to herein as respiratory device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

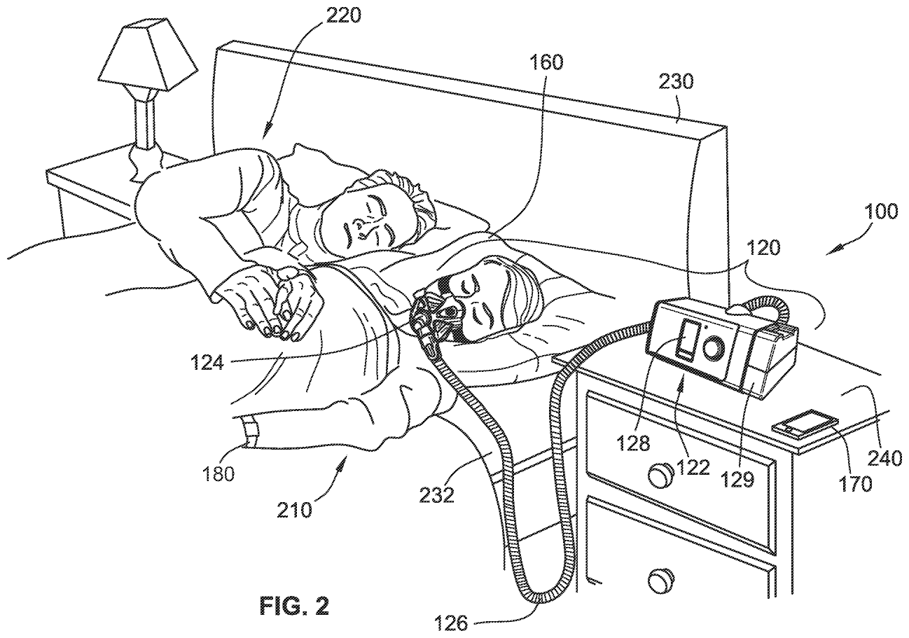

As shown in FIG. 2, in some implementations, the user interface 124 is a facial mask that covers the nose and mouth of the user. Alternatively, the user interface 124 can be a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score, the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user.

The respiratory system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example, physiological data, audio data, or both. Physiological data generated by one or more of the sensors 130 can be used by the control system 110 to determine a sleep-wake signal associated with a user during a sleep session and one or more sleep-related parameters. The sleep-wake signal can be indicative of one or more sleep states and/or sleep stages, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user enters the bed, a time that the user exits the bed, a time that the user attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory device 122, the use interface 124, the conduit 126, or the user device 170.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141, as described in, for example, WO 2018/050913 and WO 2020/104465, each of which is hereby incorporated by reference herein in its entirety. In such implementations, the speaker 142 generates or emits sound waves at a predetermined interval and/or frequency and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, pressure settings of the respiratory device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state or sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, the activity tracker 180, or any combination thereof. For example, the microphone 140 and speaker 142 is integrated in and/or coupled to the user device 170 and the pressure sensor 130 and/or flow rate sensor 132 are integrated in and/or coupled to the respiratory device 122. In some implementations, at least one of the one or more sensors 130 is not coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The user device 170 (FIG. 1) includes a display 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display 172 is generally used to display image(s) including still images, video images, or both. In some implementations, the display 172 acts as a human-machine interface (HMI) that includes a graphic user interface (GUI) configured to display the image(s) and an input interface. The display 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

In some implementations, the system 100 also includes an activity tracker 180. The activity tracker 180 is generally used to aid in generating physiological data associated with the user. The activity tracker 180 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 180 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 180 is coupled (e.g., electronically or physically) to the user device 170.

In some implementations, the activity tracker 180 is a wearable device that can be worn by the user, such as a smartwatch, a wristband, a ring, or a patch. For example, referring to FIG. 2, the activity tracker 180 is worn on a wrist of the user 210. The activity tracker 180 can also be coupled to or integrated a garment or clothing that is worn by the user. Alternatively still, the activity tracker 180 can also be coupled to or integrated in (e.g., within the same housing) the user device 170. More generally, the activity tracker 180 can be communicatively coupled with, or physically integrated in (e.g., within a housing), the control system 110, the memory 114, the respiratory system 120, and/or the user device 170.

Referring back to FIG. 1, while the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory system 120, at least one of the one or more sensors 130, and the user device 170. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

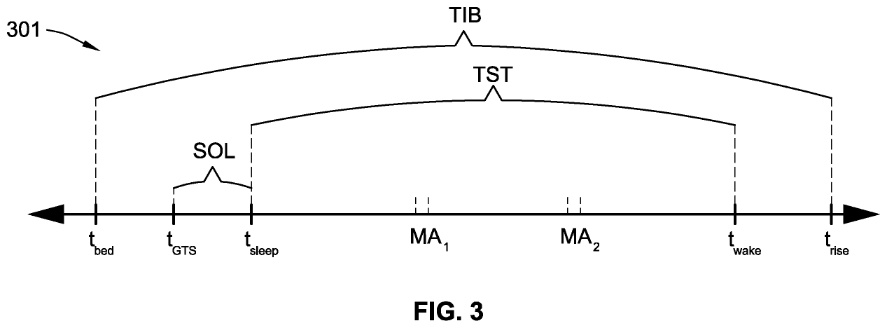

Referring to FIG. 3, an exemplary timeline 301 for a sleep session is illustrated. The timeline 301 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (teed) and ending at the wake-up time (t_{wake}). In some implementations, a sleep session is defined as starting at the initial sleep time (tₛₗₑₑₚ) and ending at the rising time (tᵣᵢₛₑ).

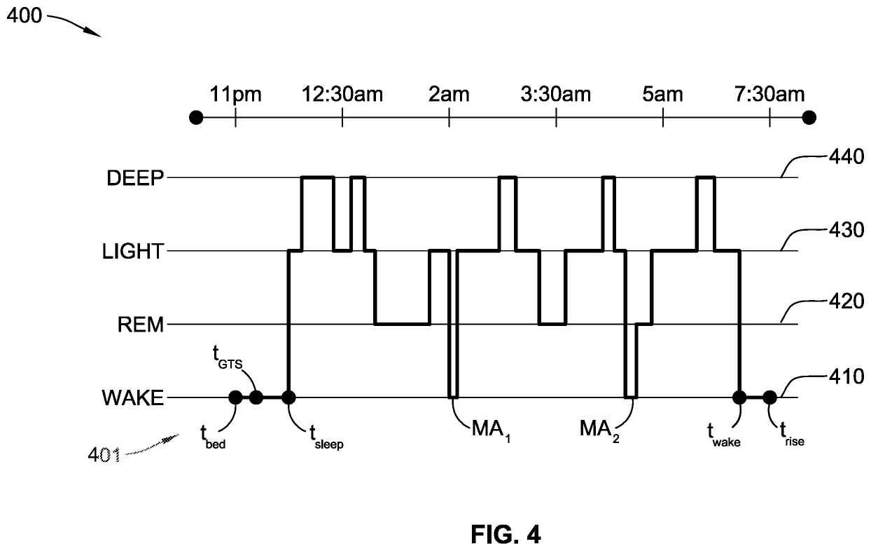

Referring to FIG. 4, an exemplary hypnogram 400 corresponding to the timeline 301 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnograph 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory device 122, data indicative of the user donning the user interface 124, etc.), or any combination thereof.

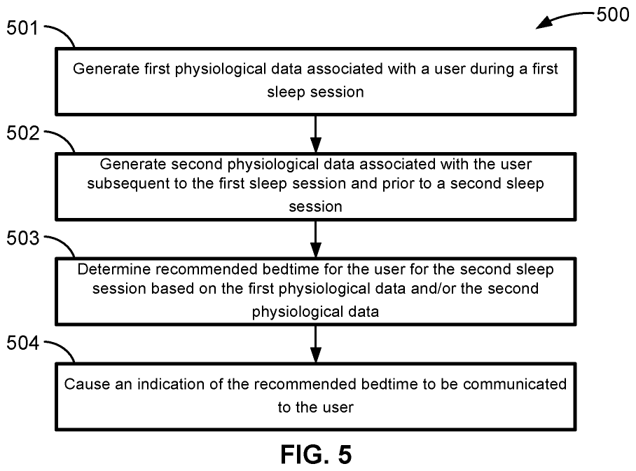

Referring to FIG. 5, a method 500 for determining a recommended bedtime for a user is illustrated. One or more steps of the method 500 can be implemented using any element or aspect of the system 100 (FIGS. 1-2) described herein.

Step 501 of the method 500 includes generating first physiological data associated with a user during at least a portion of a first sleep session. For example, step 501 can include generating or obtaining first physiological data during the first sleep session using at least of the one or more sensors 130 (FIG. 1). In some implementations, the first physiological data is generated using the acoustic sensor 141 or the RF sensor 147 described above, which are coupled to or integrated in the user device 170. In other implementations, the first physiological data is generated or obtained using the pressure sensor 132 and/or the flow rate sensor 134 (FIG. 1), which are coupled to or integrated in the respiratory device 122. Information describing the first physiological data generated during step 501 can be stored in the memory device 114 (FIG. 1).

Step 501 can include generating first physiological data during a segment of the first sleep session, during the entirety of the first sleep session, or across multiple segments of the first sleep session. For example, step 501 can include generating the first physiological data continuously or discontinuously during between about 1% and 100% of the first sleep session, at least 10% of the first sleep session, at least 30% of the first sleep session, at least 50% of the first sleep session, at least 90% of the first sleep session, etc.

Step 502 of the method 500 includes generating second physiological data associated with the user subsequent to the first sleep session and prior to a second sleep session. For example, step 502 can include generating or obtaining second physiological data during subsequent to the first sleep session and prior to a second sleep session using at least of the one or more sensors 130 (FIG. 1). Information describing the second physiological data generated during step 502 can be stored in the memory device 114 (FIG. 1).

In some implementations, the first physiological data from (step 501) is generated using a first one of the sensors 130 and the second physiological data (step 502) is generated using a second of the sensors 130 that is separate and distinct from the first sensor. In such implementations, the first sensor and the second sensor can be different types of sensors (e.g., the first sensor is an acoustic sensor that is the same as, or similar to, the acoustic sensor 141, and the second sensor is a motion sensor that is the same as, or similar to, the motion sensor 138). Alternatively, the first sensor and the second sensor can be the same sensor. As described herein, while the system 100 (FIG. 1) is shown as including one user device 170, in some implementations, the system 100 can include any number of user devices that are the same as, or similar to, the user device 170. In such implementations, a first one of the sensors 130 for generating the first physiological data (step 501) can be coupled to or integrated in a first user device (e.g., a smartphone), while a second one of the sensors 130 for generating the second physiological data (step 502) can be coupled to or integrated in a second user device (e.g., a wearable device, such as a smart watch) that is separate and distinct from the first user device.

In some implementations, the first sleep session and the second sleep session are immediately successive sleep sessions. For example, the first sleep session can begin Monday evening and end Tuesday morning, while the second sleep session begins on Tuesday evening and ends on Wednesday morning. Alternatively, there can be one or more additional, intervening sleep sessions between the first sleep session and the second sleep session such that the first sleep session and the second sleep session are not immediately successive sleep sessions. For example, the first sleep session can begin Monday evening and end Tuesday morning, an intermediate or intervening sleep session begins on Tuesday evening and ends on Wednesday morning, and the second sleep session begins on Wednesday evening and ends on Thursday morning.

Step 502 of the method 500 can include generating the second physiological data associated with the user during the entire duration between the first sleep session and the second sleep session, or during one or more segments or portion of the duration between the first sleep session and the second sleep session. For example, step 502 can include generating the second physiological data during between about 1% and about 99% of the duration between the first sleep session and the second sleep session, at least 10% of the duration between the first sleep session and the second sleep session, at least 30% of the duration between the first sleep session and the second sleep session, at least 50% of the duration between the first sleep session and the second sleep session, at least 90% of the duration between the first sleep session and the second sleep session, at least about 2 hours, at least about 5 hours, at least about 8 hours, at least about 10 hours, etc.

Step 503 of the method 500 includes determining a recommended bedtime for the user for the second sleep session based at least in part on the first physiological data, the second physiological data, or both. The control system 110 can analyze the first physiological data and/or the second physiological data stored in the memory device 114 to determine the recommended bedtime. Generally, the recommended bedtime is determined so that the user will be more likely to experience quality sleep during the second sleep session. For example, the recommended bedtime for the second sleep session can be determined by determining the bedtime for which the user will have the highest predicted sleep score for the second sleep session. The sleep scores referred to herein are exemplified by the ones described in International Publication No. WO 2015/006364 (see, e.g., paragraphs [0278]-[0285]), which is hereby incorporated by reference herein in its entirety. Alternative definitions are also possible.

As described herein, the first physiological data (step 501) was generated during the first sleep session. Thus, the first physiological data can be used to determine the recommended bedtime for the second sleep session based on sleep-related parameters associated with the first sleep session. For example, if the bedtime for the first sleep session was 9:45 PM and the first physiological data indicates that the user had a sleep-on-set latency (SOL) greater than a predetermined threshold (e.g., greater than about 20 minutes, greater than about 45 minutes, greater than about 1 hours, etc.), the recommended bedtime for the second sleep session can be later than the first sleep session (e.g., 10:30 PM) so that the user is more tired at bedtime and more likely to fall asleep quicker.

Additionally, as described herein, the second physiological data (step 502) was generated subsequent to the first sleep session but before the second sleep session. Thus, the second physiological data can be used to determine the recommended bedtime based on the user's activity levels during the day prior to the second sleep session. For example, if the second physiological data indicates that the user was fatigued during the data (e.g., due to lack of enough sleep during the first sleep session), the recommended bedtime for the second sleep session can be earlier than the bedtime for the first sleep session. As another example, if the second physiological data indicates that the user had high activity levels during the day (e.g., exercise) and/or high stress levels during the day, the recommended bedtime for the second sleep session can be earlier than the bedtime for the first sleep session.

In some implementations, step 503 includes using a machine learning algorithm to determine the recommended bedtime for the user for the second sleep session. For example, step 503 can include using neural networks (e.g., shallow or deep approaches) to determine the recommended bedtime. Step 503 can include using supervised machine learning algorithms/techniques and/or unsupervised machine learning algorithms/techniques.

Generally, the machine learning algorithm receives one or more inputs and determines as an output the recommended bedtime or a recommended bedtime window (e.g., including an optimal window and acceptable margins around the optimal window). The inputs to the machine learning algorithm can include, for example, one or more of: prior sleep session data (e.g., for one or more sleep sessions, sleep state and wake epochs, sleep efficiency, sleep score, sleep onset latency, wake after sleep onset, sleep duration, percentage of each state, number of sleep cycles, etc.), age, gender, a subjective indication of fatigue and/or sleepiness, chronic conditions, insomnia, hyperarousal, participation in active cognitive behavioral therapy (CBT) program, eating times and quantities (e.g., including calories and types of food), exercise (e.g., as indicated by a number of steps, intensity, activity types, etc.), any SDB and any associated SDB treatments (e.g., MRD, PAP, etc.), percentages of REM sleep per sleep session, a comparison between deep sleep against personal normative values or population normative values, a number and duration of conscious and unconscious awakenings, medication taken, any leak if using the respiratory therapy system (e.g., such as mouth or general unintentional leak), breathing parameters, day of week, other schedule information (e.g., such as meetings, transport, etc. in a work or personal calendar), or any combination thereof. The inputs can also include similar such information from a bed partner.

Step 504 of the method 500 includes causing an indication of the recommended bedtime determined during step 503 to be communicated to the user. The indication of the recommended bedtime can be communicated to the user at the recommended bedtime, or before the recommended bedtime (e.g., 30 seconds before, 5 minutes before, 20 minutes before, 1 hour before, 3 hours before, etc.). The indication may further include sleep hygiene recommendations, such as avoiding (further) caffeine, alcohol, electronic devices, excessive bedroom light, etc.

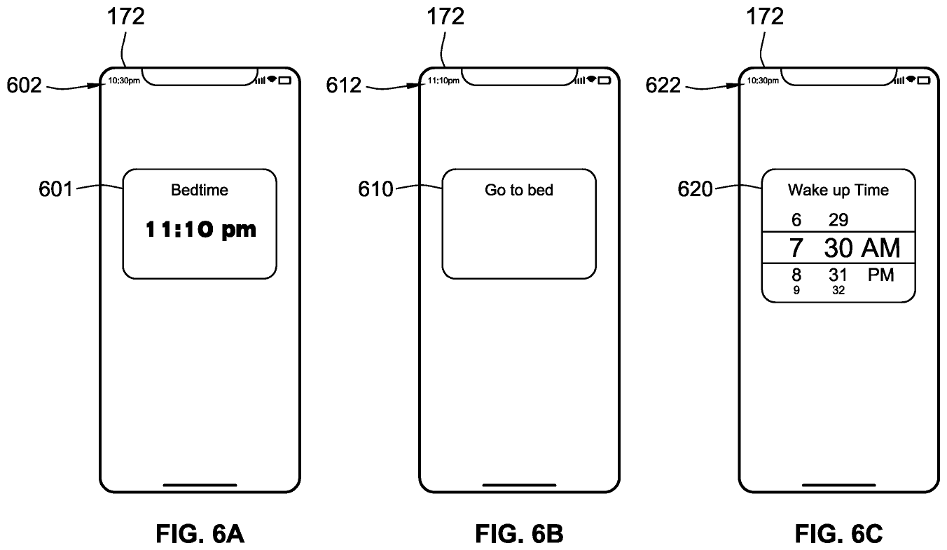

Referring to FIG. 6A, in some implementations, step 504 includes causing a visual indication of the recommended bedtime to be communicated to the user via the display device 172 of the user device 170 (FIG. 1) before the recommended bedtime. As shown in FIG. 6A, an indication 601 of the recommended bedtime is displayed on the display device 172 of the user device 170 before the recommended bedtime. The indication 601 includes alphanumeric text to communicate the recommended bedtime (in this example, 11:10 PM) to the user via the display device 172. A current time 602 (in this example, 10:30 PM) can also be displayed on the display device 172 along with the indication 601 of the recommended bedtime.

In some implementations, step 504 additionally or alternatively includes causing a visual indication of the recommended bedtime to be communicated to the user via the display device 172 of the user device 170 (FIG. 1) at the recommended bedtime. Referring to FIG. 6B, an indication 610 of the recommended bedtime is displayed on the display device 172 of the user device 170 at the recommended bedtime (in this example, 11:10 PM). The indication 610 includes alphanumeric text for communicating the recommended bedtime to the user. A current time 612 (in this example, 11:10 PM) can also be displayed on the display device 172 along with the indication 610.

In other implementations, step 503 of the method 500 additionally or alternatively includes causing an audio indication of the recommended bedtime to be communicated to the user (e.g., via the speaker 142). In such implementations, the audio indication can include speech that communicates the recommended bedtime to the user (e.g., “go to bed at 11:10 PM tonight,” “go to bed in 20 minutes,” “go to bed now” etc.) and/or other sound(s) communicating the recommended bedtime to the user.

In some implementations, step 504 of the method 500 includes causing the indication of the recommended bedtime to be communicated to the user at a reminder time that is before the recommended bedtime. Communicating the recommended bedtime at the reminder time (e.g., rather than only at the recommended bedtime) is advantageous because the user has advance notice of the recommended bedtime and can begin winding down and getting ready so that the user is ready to go to bed at the recommended bedtime. For example, referring to FIG. 6A, the indication 601 of the recommended bedtime (in this example, 11:10 PM) is displayed on the display device 172 before the recommended bedtime, as shown by the current time 602 (in this example, 10:30 PM).

In some implementations, the reminder time is determined based solely on the recommended bedtime such that the difference between the reminder time and the recommended bedtime is constant over a series of sleep sessions. The reminder time can be between about 30 seconds and about 6 hours before the recommended bedtime, between about 5 minutes and about 2 hours before the recommended bedtime, between about 15 minutes and about 1 hour before the recommended bedtime, between about 20 minutes and about 45 minutes before the recommended bedtime. For example, in such implementations, if the difference is set at 30 minutes, the reminder time for a recommended bedtime of 11:30 PM will be 11:00 PM and the reminder time for a recommended bedtime of 9:30 PM will be 9:00 PM.

In other implementations, the reminder time is determined in the same or similar manner as the recommended bedtime based at least in part on the first physiological data (step 501), the second physiological data (step 502), previous reminder times, or any combination thereof. For example, if previously recorded data indicates that the user, on average, takes 40 minutes to go bed, the reminder time for the next sleep session can be set at 40 minutes prior to the recommended bedtime. As another example, if the reminder time for the first sleep session was set at 30 minutes before the recommended bedtime for the first sleep session, but the user did not go to bed until 10 minutes after the recommended bedtime, the reminder time for the second sleep session can be set at 40 minutes before the recommended bedtime for the second sleep session.

In some implementations, the method 500 includes receiving, from the user, information indicative of a desired wake-up time following the second sleep session. The control system 110 can cause the user to be prompted to provide the desired wake-up time. For example, referring to FIG. 6C, the control system 110 can a prompt 620 to be displayed on the display device 172 of the user device 170 (FIG. 1) that provides an interface for the user to specify the desired wake-up time. In the example of FIG. 6A, the user can select the desired wake-up time using a touchscreen by scrolling to select the desired hour, minute, and AM or PM. Alternatively, the user can input the desired wake-up time using an alphanumeric keyboard (e.g., that is display on the touchscreen display) or speech-to-text. In implementations of the method 500 including receiving the desired wake-up time, step 503 can include adjusting the recommended bedtime based at least in part on the received desired wake-up time. For example, the recommended bedtime can be adjusted so that the user can sleep for a predetermined sleep duration (e.g., at least 6 hours, at least 7 hours, at least 8 hours, etc.). For example, if the recommended bedtime for the second sleep session is determined to be 11:00 PM based on the physiological data and the predetermined duration is 7 hours, but the desired wake-up time is 5:00 AM, the recommended bedtime can be adjusted to 10:00 PM so that the user can sleep for 7 hours.

In such implementations of the method 500 including receiving the desired wake-up time, the method 500 can also include causing an alarm to be set for the desired wake-up time. For example, the control system 110 can cause the user device 170 to set an alarm for the desired wake-up time so that the user does not need to manually set another alarm using the user device 170 or another device.

In some implementations, step 503 of the method 500 also includes determining a recommended wake-up time and/or a recommended sleep duration in addition to the recommended bedtime. The recommended wake-up time and/or the recommended sleep duration can be determined in the same or similar manner as the recommended bedtime (e.g., using a machine learning algorithm). In such implementations, the method 500 can also include causing an alarm to be generated (e.g., on the user device 170) at the recommended wake-up time so that the user does not need to manually set an alarm to wake up at the recommended wake-up time. Further, in such implementations, the method 500 can also include causing one or more calendar events to be displayed on the display device 172 of the user device 170 (FIG. 1) along with the recommended wake-up time. The user can then view their schedule for the next day and accept or decline (e.g., modify) the recommended wake-up time. For example, if the recommended wake-up time is 7:30 AM but the user needs to wake up earlier for an event (e.g., work, a flight, etc.), the user can be reminded of the event via the calendar and either accept the recommended wake-up time (e.g., by selecting a user-selectable element that is displayed on the display device 172) or decline or modify the wake-up time (e.g., using the prompt 620 in FIG. 6C).

In some implementations, the method 500 also includes causing a second indication indicative of one or more recommended user actions to be communicated to the user. The recommended user action(s) can be communicated to the user along with the indication of the recommended bedtime (simultaneous with step 504), before the indication of the recommended bedtime is communicated to the user (before step 504), or after the indication of the recommended bedtime is communicated to the user (after step 504). Generally, the recommended user action is selected to aid the user in falling asleep at the recommended bedtime. For example, the recommended user action(s) can include recommending that the user turn off a television (e.g., to reduce ambient noise and light), recommending that the user lower an ambient audio intensity (e.g., lower the volume on a TV), recommending that the user lower an ambient light intensity (e.g., dim or turn off lights in the bedroom), recommending that the user brush their teeth, recommending that the user remove eyewear (e.g., contacts or glasses), recommending that the user wear the user interface 124 of the respiratory system 120 (FIG. 1), or any combination thereof.

In some implementations, the method 500 also includes causing one or more environmental parameters to be modified for the second sleep session. The environment parameter(s) can be modified at the same time that the recommended bedtime is communicated to the user (step 503), before the recommended bedtime is communicated to the user, or after the recommended bedtime is communicated to the user. For example, the control system 110 (FIG. 1) can be communicatively coupled to one or more user devices to modify an ambient light intensity (e.g., by dimming or turning off lights), modify an ambient audio intensity (e.g., by turning off a TV or turning down the volume), modify an ambient temperature (e.g., by changing one or more thermostat settings), or any combination thereof.

In some implementations, the method 500 includes causing one or more functions of a user device to be restricted at or before the recommended bedtime to aid or encourage the user in to go to bed at the recommended bedtime. For example, the control system 110 (FIG. 1) can restrict one or more functions of the user device 170 (e.g., a smartphone, a tablet, a smart TV, a wearable device, a laptop, a television, etc.) such as web browsing or surfing, access to social media applications, access to entertainment applications (e.g., video streaming services), access to games, etc., while allowing other functions (e.g., setting an alarm, phone calls, text messages, viewing calendar entries, etc.).

In some implementations, the method 500 includes receiving user-reported feedback from the user subsequent to the first sleep session. In such implementations, step 503 can include determining the recommended bedtime based at least in part on the user-reported feedback. The user-reported feedback can include, for example, a subjective sleep score for the first sleep session (e.g., poor, average, good, excellent, etc.), a subjective fatigue level (e.g., tired, average, rested), a subjective stress level (e.g., low, average, high), a subjective health status (e.g., healthy, unhealthy, sick, etc.), or any combination thereof, following the first sleep session.

In some implementations, the method 500 includes receiving personal information associated with the user and step 503 includes determining the recommended bedtime for the second sleep session based at least in part on the received personal information. The personal data can include demographic data, such as, for example, information indicative of an age of the user, a gender of the user, a race of the user, an employment status of the user, an educational status of the user, a socioeconomic status of the user, a recent life event (e.g., change in relationship status, birth of child, death in family etc.), information as to whether the user has a family history of sleep-related disorders or any combination thereof. The personal data can also include medical data, such as, for example, information (e.g., medical records) indicative of one or more medical conditions that the user has been diagnosed with, medication usage, or both. The personal data can also include information provided by a third party (e.g., medical records from a medical provider, a questionnaire or feedback from a family member or friend associated with the user, etc.).

In some implementations, the method 500 includes determining one or more sleep-related parameters for the first sleep session based on the first physiological data (step 501) and/or the second physiological data (step 502). The sleep-related parameters can include a respiration signal, a respiration rate, an inspiration amplitude, an expiration amplitude, an inspiration-expiration ratio, an occurrence of one or more events, a number of events per hour, a pattern of events, a sleep state, a sleep stage, or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof.

One or more of the steps of the method 500 described herein can be repeated one or more time for additional sleep sessions (e.g., a third sleep session, a fourth sleep session, a tenth sleep session, etc.). For example, steps 501 and 502 can be repeated to generate third physiological data associated with the user during the second sleep session and generate fourth physiological data associate with the user during a third sleep session that is subsequent to the second sleep session. Step 503 can then be repeated to determine a recommended bedtime for the third sleep session based at least in part on the third physiological data and/or the fourth physiological data. In some implementations, the method 500 includes adjusting the recommended bedtime, the reminder time, or both from the second sleep session for the third sleep session. Step 504 can be repeated such that the recommended bedtime for the third sleep session can be communicated to the user in the same or similar manner as described herein for the second sleep session (e.g., the control system 110 causes an indication of the recommended bedtime for the third sleep session to be communicated to the user via the user device 170).

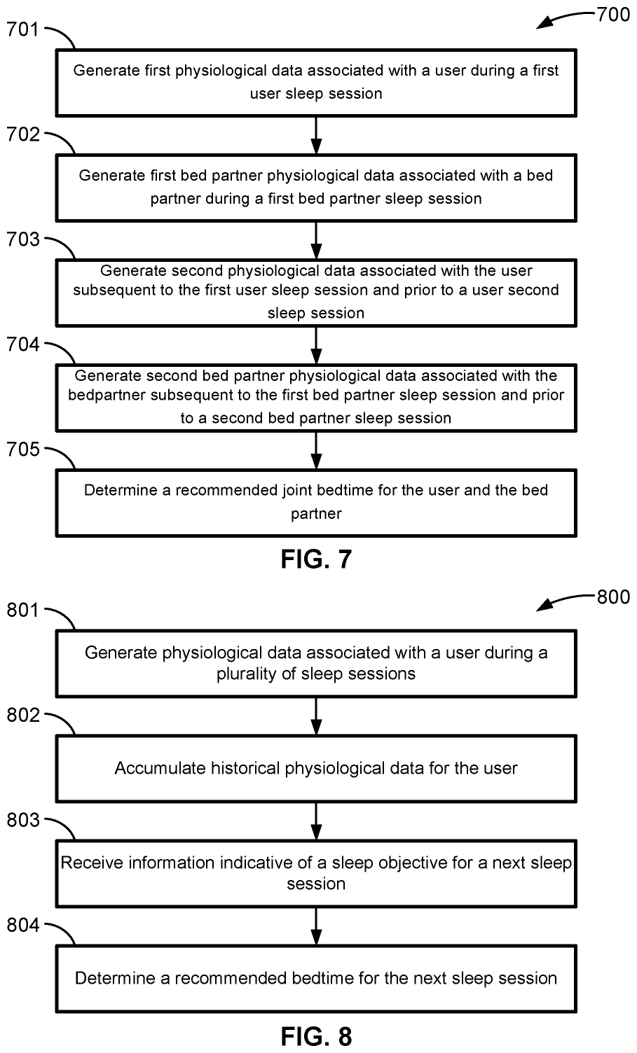

Referring to FIG. 7, a method 700 for determining a joint recommended bedtime for a user and a bed partner is illustrated. One or more steps of the method 700 can be implemented using any element or aspect of the system 100 (FIGS. 1-2) described herein.

Step 701 of the method 700 is the same as, or similar to, step 501 of the method 500 (FIG. 5) and includes generating first physiological data associated with a user during a first user sleep session (hereinafter, first user physiological data).

Step 702 of the method 700 includes generating first bed partner physiological data associated with a bed partner of the user during a first bed partner sleep session. For example, referring to FIG. 2, the user 210 is in the bed 230 with a bed partner 220. Step 702 is the same as, or similar to, step 701, except that step 702 includes generating physiological data (hereinafter, first bed partner physiological data) for the bed partner 220 rather than the user 210. Like the first user physiological data (step 701), the first bed partner physiological data (step 702) can be stored in the memory device 114 (FIG. 1).

In some implementations, the generation of the first bed partner physiological data (step 702) is simultaneous with the generation of the first user physiological data (step 701) such that the generation of the first user physiological data (step 701) overlaps with the generation first bed partner physiological data (step 701). In other implementations, the generation of the first user physiological data (step 701) partially overlaps the generation of the first bed partner physiological data (step 701) (e.g., the generation of the first user physiological data (step 701) begins before or after the generation of the first bed partner physiological data (step 702), or vice versa) such that the first user sleep session at least partially overlaps with the first bed partner sleep session. Alternatively, the generation of the first user physiological data (step 701) does not overlap with the generation of the first bed partner physiological data (step 701) such that the first user sleep session does not overlap with the first bed partner sleep session (e.g., the first user sleep session is on a Monday night and the first bed partner sleep session is on Tuesday night).

The first bed partner physiological data (step 702) and the first user physiological data (step 701) can be generated using the same sensor(s) from the one or more sensors 130 (FIG. 1) described herein, or different ones of the sensors 130. For example, in FIG. 2, the user 210 is wearing the user interface 124 of the respiratory system 120 and the bed partner 220 is not. Thus, in this example, the first user physiological data (step 701) can be generated using one or more of the sensors 130 that are coupled to or integrated in the respiratory system 120, while the second user physiological data (step 702) is generated by another one of the sensors 130 that is external to the respiratory system 120 (e.g., one or more sensors that are coupled to or integrated in the user device 170 that is positioned on the nightstand 240 in FIG. 2).

Step 703 of the method 700 is the same as, or similar to, step 502 of the method 500 (FIG. 5) and includes generating second physiological data associated with the user subsequent to the first user sleep session and prior to a user second sleep session (hereinafter, the second user physiological data).

Step 704 of the method 700 includes generating second physiological data associated with the bed partner subsequent to the first bedpartner sleep session and prior to a bedpartner second sleep session (hereinafter, second bed partner physiological data). Generally, the user and the bed partner will not be in close proximity during the entire day between sleep sessions (e.g., both go to work in different locations). Thus, step 703 can include using a first sensor and step 704 can include using a second sensor that is different than the first sensor. As described herein, the system 100 can include a plurality of user devices that are the same as, or similar to, the user device 170 (FIG. 1). Thus, the first sensor for the second user physiological data (step 703) can be coupled to or integrated in a first user device that is associated with the user (e.g., a smartphone), while the second sensor for the second bed partner physiological data (step 704) can be coupled to or integrated in a second user device that is associated with the bed partner.

Step 705 of the method 700 includes determining a recommended joint bedtime for the user and the bed partner based at least in part on the first user physiological data, the first bed partner physiological data, the second user physiological data, the second bedpartner physiological data, or any combination thereof. The user 210 and the bed partner 220 may generally have different sleep schedules. For example, the bed partner 220 may tend to go to bed later than the user 210 (e.g., after the user 210 is already asleep), which can disrupt the sleep of the user 210. The recommended joint bedtime is generally selected to attempt to achieve higher quality sleep for both the user 210 and the bed partner 220 so that they go to bed at the same time and one does not disrupt the sleep of the other.

In some implementations, step 705 includes determining a recommended bedtime for the user based at least in part on the first user physiological data (step 701), the second user physiological data (step 703), or both and determining a recommended bedtime for the bed partner based at least in part on the first bed partner physiological data (step 702), the second bed partner physiological data (step 704) or both. In such implementations, the recommended joint bedtime can be determining by averaging the recommended bedtime for the user and the recommended bedtime for the bed partner. For example, if the recommended bedtime for the user is 10:20 PM and the recommended bedtime for the bed partner is 10:40 PM, the recommended joint bedtime is 10:30 PM. In this manner, any deviation from the individual recommended bedtime is shared by the user and the bed partner to minimize the impact(s) from deviating from the individual recommended bedtime.

Step 705 can include using an algorithm (e.g., machine learning algorithms) to determine the recommended joint bedtime. The algorithm can be used to determine a joint bedtime that will have the lowest predicted negative impact on the user and the bed partner. For example, if the recommended individual bedtime for the user is 11:00 PM and the recommended individual bedtime for the bed partner is 10:00 PM, averaging these will result in a recommended joint bedtime at 10:30 PM. However, the algorithm can determine that shifting the individual bedtime for the user by 30 minutes will have a greater impact on the user (e.g., in terms of a predicted sleep score) than shifting the individual bedtime for the bedpartner by 45 minutes from the individual bedtime for the bedpartner. Thus, in this example, the recommended joint bedtime will be 10:45 PM. The algorithm can receive any of the inputs described above and determine as an output the recommended joint bedtime.

The method 700 can also include causing the recommended joint bedtime to be communicated to the user, the bed partner, or both. The recommended joint bedtime can be communicated in the same or similar manner as the recommended bedtime in step 504 of the method 500 (FIG. 5) described above. For example, a first visual indicator of the recommended joint bedtime can be displayed on a user device associated with the user and a second visual indicator of the recommended joint bedtime can be displayed on a user device associated with the bed partner.

In some implementations, the method 700 also includes determining a recommended joint wake-up time for the user and the bed partner. For example, the bed partner 220 may tend to get up earlier in the morning than the user 210, which can disturb or interrupt the sleep of the user 210. In such implementations, the method 700 includes determining the recommended joint wake-up time in the same or similar manner as the recommended joint bedtime described above (step 705).

Referring to FIG. 8, a method 800 for determining a recommended bedtime for a user is illustrated. One or more steps of the method 800 can be implemented using any element or aspect of the system 100 (FIGS. 1-2) described herein.

Step 801 of the method 800 is similar to step 501 of the method 500 (FIG. 5) described herein and includes generating first physiological data associated with a user during at least a portion of a plurality of sleep sessions. The plurality of sleep sessions includes one or more pairs of sleep sessions. Each of the one or more pairs of sleep sessions includes a first sleep session and a second sleep session that is subsequent to the first sleep session.

In some implementations, the first sleep session and the second sleep session are successive sleep sessions (e.g., the first sleep session begins in the evening on a first day and ends in the morning on a second day and the second sleep session begins in the evening on the second days). In other implementations, the first sleep session and the second sleep session and not immediately successive sleep sessions (e.g., the first sleep session beings in the evening on a first day and ends in the morning on a second day, while the second sleep session begins on a fourth day that is subsequent to the second day).

Step 802 of the method 800 includes accumulating historical physiological data for the user for the plurality of sleep sessions. The historical physiological data can be stored in the memory device 114 (FIG. 1) of the system 100. In some implementations, step 802 includes accumulating historical physiological data for a predetermined number of sleep sessions (e.g., 7 sleep sessions, 14 sleep sessions, 30 sleep sessions, 90 sleep sessions, 180 sleep sessions, 365 sleep sessions, 1,000 sleep session, etc.). In such implementations, physiological data for older sleep sessions can be deleted automatically once the predetermined number has been reached.

Step 803 of the method 800 includes receiving information indicative of a sleep objective for a next sleep session. The sleep objective can include, for example, a desired sleep score for the next sleep session, a desired subjective sleep score (e.g., poor, good, excellent), a desired sleep duration (e.g., at least 6 hours, at least 7 hours, at least 8 hours, etc.), a desired wake-up time, or any combination thereof.

Step 804 of the method 800 includes determining a recommended bedtime for the next sleep session based at least in part on the accumulated historical physiological data and the received sleep objective. For example, step 804 can include training a machine learning algorithm using the accumulated historical physiological data (step 802) such that the machine learning algorithm can receive as an input information indicative of the sleep objective (step 803) and output a recommended bedtime for the next sleep session. The recommended bedtime is determined to aid the user in achieving the desired sleep objective. The recommended bedtime (step 804) can be communicated to the user in the same or similar manner as the recommended bedtime described above for the method 500 (FIG. 5) (e.g., by causing an indicator of the recommended bedtime to be communicated to the user).

In some implementations, the method 800 includes determining a recommended wake-up time for the next sleep session based at least in part on the accumulated historical physiological data (e.g., using a trained machine learning algorithm).

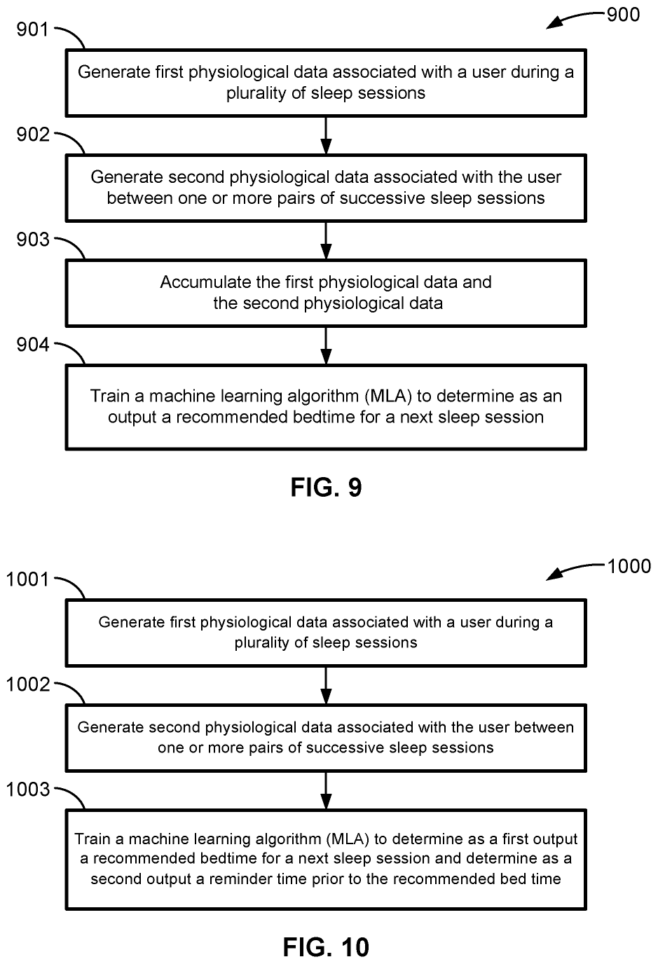

Referring to FIG. 9, a method 900 for determining a recommended bedtime for a user is illustrated. One or more steps of the method 900 can be implemented using any element or aspect of the system 100 (FIGS. 1-2) described herein.

Step 901 of the method 900 includes generating first physiological data associated with a user during a plurality of sleep sessions. The plurality of sleep sessions includes one or more pairs of sleep sessions. Each of the one or more pairs of sleep sessions includes a first sleep session and a second sleep session that is subsequent to the first sleep session. In some implementations, the first sleep session and the second sleep session are successive sleep sessions (e.g., the first sleep session begins in the evening on a first day and ends in the morning on a second day and the second sleep session begins in the evening on the second days). In other implementations, the first sleep session and the second sleep session and not immediately successive sleep sessions (e.g., the first sleep session beings in the evening on a first day and ends in the morning on a second day, while the second sleep session begins on a fourth day that is subsequent to the second day).

Step 902 of the method 900 includes generating second physiological data associated with the user between one or more pairs of successive sleep sessions.

Step 903 of the method 900 is similar to step 802 of the method 800 (FIG. 8) and includes accumulating the first physiological data and the second physiological data for the plurality of the sleep sessions. The accumulated first physiological data and the second physiological data can be stored in the memory device 114 (FIG. 1) of the system 100. In some implementations, step 903 includes accumulating historical physiological data for a predetermined number of sleep sessions (e.g., 7 sleep sessions, 14 sleep sessions, 30 sleep sessions, 90 sleep sessions, 180 sleep sessions, 365 sleep sessions, 1,000 sleep session, etc.). In such implementations, physiological data for older sleep sessions can be deleted automatically once the predetermined number has been reached.

Step 904 of the method 900 includes training a machine learning algorithm (MLA) such that the MLA receives as an input second physiological data and determines as an output a recommended bedtime for a next sleep session. The machine learning algorithm can be trained (e.g. supervised or unsupervised) using the accumulated historical physiological data stored in the memory device 114 (FIG. 1). The recommended bedtime can be communicated to the user in the same or similar manner as step 504 of the method 500 (FIG. 5).

In some implementations of the method 900, the machine learning algorithm is also trained to determine as a second output a recommended wake-up time for the user for the next sleep session. In such implementations, the method 900 can also include causing an alarm to be generated at the recommended wake-up time (e.g., on the user device 170 (FIG. 1)) so that the user does not need to manually set an alarm for the next sleep session.

Referring to FIG. 10, a method 1000 for determining a recommended bedtime for a user is illustrated. One or more steps of the method 1000 can be implemented using any element or aspect of the system 100 (FIGS. 1-2) described herein.

Step 1001 of the method 1000 is similar to step 501 of the method 500 (FIG. 5) described herein and includes generating first physiological data associated with a user during a plurality of sleep sessions. The plurality of sleep sessions includes one or more pairs of sleep sessions. Each of the one or more pairs of sleep sessions includes a first sleep session and a second sleep session that is subsequent to the first sleep session. In some implementations, the first sleep session and the second sleep session are successive sleep sessions (e.g., the first sleep session begins in the evening on a first day and ends in the morning on a second day and the second sleep session begins in the evening on the second days). In other implementations, the first sleep session and the second sleep session and not immediately successive sleep sessions (e.g., the first sleep session beings in the evening on a first day and ends in the morning on a second day, while the second sleep session begins on a fourth day that is subsequent to the second day).

Step 1002 of the method 1000 is the same as, or similar to, step 902 of the method 900 (FIG. 9) and includes generating second physiological data associated with the user between one or more pairs of sleep sessions.

In some implementations, step 1002 includes continuously generating the second physiological data between the pairs of the sleep sessions (e.g., during 100% of the time between the first sleep session and the second sleep session). In other implementations, step 1002 includes continuously or discontinuously generating between the pairs of sleep sessions, for example, between about 1% and 99% of the time between the first sleep session and the second, subsequent sleep session, at least about 10% of the time between the first sleep session and the second sleep session, at least about 25% of the time between the first sleep session and the second sleep session, at least 66% of the time between the first sleep session and the second sleep session, at least 90% of the time between the first sleep session and the second sleep session, etc.

Step 1003 of the method 1000 includes training a machine learning algorithm (MLA) such that the MLA receives as an input second physiological data and determines as a first output a recommended bedtime for a next sleep session and determine as a second output a reminder time that is before the recommended bed time. More generally, the machine learning algorithm can receive any of the inputs described above. As described herein, the recommended bedtime for the next sleep session can be communicated to the user using an indicator (e.g., a visual indicator, an audio indicator, or both) at the reminder time that is before the recommended bedtime.

The systems and methods described herein can be used to assist individuals suffering from adverse physiological conditions (e.g., lower performance, slower reaction time, increased risk of depression, anxiety disorders, etc.) and adverse physiological conditions (e.g., high blood pressure, increased risk of heart disease, poor immune system function, obesity, etc.) by recommending an optimal time for the user to go to bed each night. These systems and methods can determine the recommended bedtime based on physiological data and/or the user's subjective feelings, which allows for a personalized recommendation. These and other benefits can reduce reliance on pharmacological therapy and its associated downsides (e.g., side effects and/or dependency) and reduce the burden on busy clinicians and sleep labs. Improved sleep can also increase the efficacy of vaccines.

One or more elements or aspects or steps, or any portion(s) thereof, from one or more of any of claims 1-107 below can be combined with one or more elements or aspects or steps, or any portion(s) thereof, from one or more of any of the other claims 1-107 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving first physiological data associated with a user during a first sleep session;
receiving second physiological data associated with the user subsequent to the first sleep session and prior to a second sleep session;
determining a recommended bedtime for the user for the second sleep session based at least in part on the first physiological data and the second physiological data, and
causing an indication of the recommended bedtime for the second sleep session to be communicated to the user via a user device before the recommended bedtime.

2. The method of claim 1, wherein the indication is caused to be displayed on a display of the user device at a reminder time that is before the recommended bedtime.

3. (canceled)

4. The method of claim 2, further comprising:
receiving third physiological data associated with the user during the second sleep session;
receiving fourth physiological data associated with the user subsequent to the second sleep session and prior to the third sleep session, and
adjusting the recommended bedtime for the user, the reminder time, or both for a third sleep session based at least in part on the third physiological data, the fourth physiological data, or both.

5. (canceled)

6. The method of claim 1, further comprising causing a second indication indicative of a recommended user action to be communicated to the user before the recommended bedtime for the second sleep session, wherein the recommended user action is to turn off a television, to lower an ambient light intensity, to lower an ambient audio intensity, to brush teeth, to remove contacts, to remove glasses, to wear an interface for a respiratory system, or any combination thereof.

7. (canceled)

8. The method of claim 1, further comprising causing a modification of an environmental parameter, a restriction on one or more functions of the user device to be enforced, a restriction on one or more functions of one or more devices to be enforced, or any combination thereof, wherein the environmental parameter includes an ambient light intensity, an ambient audio intensity, an ambient temperature, or any combination thereof.

9-10. (canceled)

11. The method of claim 1, wherein the first physiological data is received from a first sensor and the second physiological data is received from a second sensor.

12. The method of claim 1, wherein (i) the first sensor is configured to generate first bed partner physiological data associated with a bed partner of the user during a first sleep session of the bed partner and (ii) at least a portion of the first sleep session of the user overlaps with at least a portion of the first bed partner sleep session and at least a portion of the second sleep session of the user overlaps with at least a portion of the second bed partner sleep session, the method further comprising:
analyzing the first bed partner physiological data to determine a first set of sleep-related parameters for the bedpartner during the first sleep session of the bed partner; and
determining a recommended bedtime for a second bedpartner sleep session of the bedpartner that is based at least in part on the first bedpartner physiological data.

13-14. (canceled)

15. The method of claim 12, further comprising determining a recommended joint bedtime for the user and the bedpartner based on the recommended bedtime for the user for the second sleep session and the recommended bedtime for the bedpartner for the fourth sleep session.

16. The method of claim 1, further comprising determining a recommended wake-up time for the user for the second sleep session based at least in part on the first physiological data, the second physiological data, or both.

17. The method of claim 16, further comprising prompting the user to indicate a desired wake-up time and causing the user device to set an alarm for the recommended wake-up time in response to receiving a selection of the user-selectable element.

18-20. (canceled)

21. The method of claim 1, further comprising:
receiving an indication from the user indicative of a desired wake-up time for the second sleep session;
modifying the recommended bed time based at least in part on the received desired wake-up time; and
automatically causing the user device to set an alarm at the desired wake-up time responsive to receiving the indication.

22-29. (canceled)

30. The method of claim 1, further comprising determining a first set of sleep-related parameters for the user during the first sleep session based at least in part on the first physiological data, wherein the first set of sleep-related parameters includes a respiration signal, a respiration rate, an inspiration amplitude, an expiration amplitude, an inspiration-expiration ratio, an occurrence of one or more events, a number of events per hour, a pattern of events, a sleep state, or any combination thereof, and wherein the one or more events include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof.

31-32. (canceled)

33. The method of claim 1, further comprising determining a first set of activity-related parameters for the user subsequent to the first sleep session and prior to the second sleep session based at least in part on the second physiological data.

34. The method of claim 33, wherein the first set of activity-related parameters includes a fatigue level of the user, an average heart rate, a maximum heart rate, an average resting heart rate, a number of burned calories, a number of steps, a number of consumed calories, food intake, liquid intake, a beverage intake, conversation activity, social activity, social medial activity, or any combination thereof.

35. The method of claim 1, further comprising determining the recommended bedtime for the second sleep session based at least in part on a comparison between a desired wake-up time for the second sleep session and an actual wake-up time for the first sleep session.

36-37. (canceled)

38. The method of claim 1, further comprising determining a recommended sleep duration for the second sleep session based on the first physiological data, the second physiological data, or both.

39-87. (canceled)

88. A method comprising:
receiving first physiological data associated with a user during a plurality of sleep sessions, the plurality of sleep sessions including one or more pairs of successive sleep sessions;
receiving second physiological data associated with the user, the second physiological data being generated between each of the one or more pairs of successive sleep sessions, the second physiological data including historical second physiological data and current second physiological data; and
determining a recommended bedtime for the user for a next sleep session using a machine learning algorithm based at least in part on the current second physiological data.

89. The method of claim 88, further comprising training the machine learning algorithm with the first physiological data and the historical second physiological data such that the machine learning algorithm is configured to (i) receive as an input the current second physiological data and (ii) determine as an output the recommended bedtime for the user for the next sleep session.

90. The method of claim 88, further comprising training the machine learning algorithm using physiological data associated with a plurality of sleep sessions associated with one or more individuals that are not the user.

91. The method of claim 88, wherein the first physiological data is generated using a plurality of sensors.

92. The method of claim 91, wherein a first portion of the first physiological data is generated using a first sensor of the plurality of sensors and a second portion of the first physiological data is generated using a second sensor of the plurality of sensors.

93. The method of claim 92, wherein the first portion of the first physiological data is associated with a first sleep session of the plurality of sleep session and the second portion of the first physiological data is associated with a second sleep session of the plurality of sleep sessions to cause the generation of an alarm at the recommended wake-up time.

94-107. (canceled)

108. The method of claim 88, further comprising determining a first set of activity-related parameters for the user subsequent to the first sleep session and prior to the second sleep session based at least in part on the second physiological data.

109. The method of claim 108, wherein the first set of activity-related parameters includes a fatigue level of the user, an average heart rate, a maximum heart rate, an average resting heart rate, a number of burned calories, a number of steps, a number of consumed calories, food intake, liquid intake, a beverage intake, conversation activity, social activity, social medial activity, or any combination thereof.
